SEQUENCE LISTING (2 sequences)

SEQ ID NO: 1 - DNA, 15 bp, Geobacillus kaustophilus
atgaacacgatgctg

SEQ ID NO: 2 - DNA, 23 bp, Geobacillus kaustophilus
gaattcttattaaaaccgccagc

(19) United States
(12) Patent Application Publication
(10) Pub. No.: US 2009/0117613 A1
(43) Pub. Date: May 7, 2009

(54) Method for redox reaction using an old yellow enzyme

(76) Inventors: Anton Glieder, Gleisdorf (AT); Matthias Schittmayer, Graz (AT); Spiros Kambourakis, Pasadena, CA (US); Simone Zach, Graz (AT)

Correspondence Address: CHRISTIE, PARKER & HALE, LLP, PO BOX 7068, PASADENA, CA, 91109-7068, US

(21) Appl. No.: 12/152,765

(22) Filed: May 15, 2008

Related U.S. Application Data
(60) Provisional application No. 60/938,057, filed on May 15, 2007.

(51) Int. Cl. C12P 33/06; C12P 7/26; C12N 9/02

(52) U.S. Cl. 435/58; 435/148; 435/189

(57) ABSTRACT

A method of selective biooxidation to non activated carbon-hydrogen bonds of substances using a Geobacillus kaustophilus ‘Old Yellow Enzyme’ is provided”. It is shown that OYEs can be used to facilitate the biooxydation of substances, such as testosterone. It is also shown that OYE can introduce double bonds to form alpha, betaalpha, beta desaturated ketones. Furthermore, it is also shown that the use of OYEs allows for the production of oxidized substances in one step reactions, which are otherwise not accessible or only accessible after complex and inefficient multi-step reactions. In addition, the OYE used shows high stability (e.g. at high temperature, or in long lasting bioconversions). An exemplary embodiment is provided showing the use of an OYE to convert testosterone to 6α-hydroxytestosterone.

### CROSS REFERENCE TO RELATED APPLICATION

Priority is claimed to provisional application Ser. No. 60/938,057, filed on May 15, 2008, entitled METHOD FOR REDOX REACTION USING AN OLD YELLOW ENZYME, the contents of which are hereby expressly incorporated herein by reference as if set forth in full.

### FIELD OF THE INVENTION

The current invention is directed to an enzymatic and/or enzyme-mediated catalysis utilizing an Old Yellow Enzyme; and more particularly to the production of oxidized or reduced substrates of interest using an Old Yellow Enzyme.

### BACKGROUND OF THE INVENTION

The oxidation and reduction of substances is of high interest because of the importance of the various metabolites that can be formed from such reactions.

Important examples of biooxidation include hydroxylation. For example, hydroxylation of testosterone by human liver microsomes allows for the formation of 1β-, 2α-/β-, 6β-, 15β-, and 16β-hydroxytestosterones, which are important metabolites for the body (Agematu et al 2006). Furthermore, these metabolites are very useful in pharmacokinetic and toxicological studies. 6 alpha and beta hydroxytestosterone could also be used as building blocks for new steroid derivatives (e.g. as new drugs).

Other important examples of bioxidation reaction include desaturation reactions. The introduction of new double bonds is highly desirable for synthetic applications. The selective introduction of double bonds at very specific positions of larger molecules can hardly be done by classical chemistry. In addition to the formation of new compounds of high interest, such as the formation of boldenone—an active pharmaceutical ingredient—from testosterone, desaturation reactions activate carbohydrates for interesting, selective chemistry, such as subsequent introduction of functional groups (e.g. hydroxyl, epoxide, halogen groups). Thus, desaturation reactions provide a new broadly applicable toolbox for organic syntheses.

Usually monooxygenases are used for these biooxidative reactions, but these compounds are not available for all substrates. Among the monooxygenases, cytochrome P450 (P450) enzymes, a superfamily of more than 160 known members, are also responsible for the biosynthesis or catabolism of steroid hormones, including the oxidative metabolism of endogenous and exogenous testosterone. (See, e.g., Wood A W, Swinney D C, Thomas P E, Ryan D E, Hall P F, Levin W, and Garland W A (1988) Mechanism of androstenedione formation from testosterone and epitestosterone catalyzed by purified cytochrome P-450b. J Biol Chem 263: 17322-17332; Yamazaki H and Shimada T (1997), Progesterone and testosterone hydroxylation by cytochromes P450, 2C19, 2C9, and 3A4 in human liver microsomes. Arch Biochem Biophys 346: 161-169; and Rendic S, Nolteernsting E, and Schänzer W (1999) Metabolism of anabolic steroids by recombinant human cytochrome P450 enzymes. J Chromatogr Biomed Appl 735: 73-83).

In monooxygenase catalyzed biooxidative reaction, β-hydroxylation at either the C6 or C16 position is the major route of testosterone oxidative metabolism. Human liver enzymes are also found to oxidize testosterone at the C17 position to form androstenedione.

However, conventional oxidation enzymes, such as P450 enzymes, are usually very unstable. In addition, the monooxygenases known to catalyze these bio-oxidative reactions are not available for all substrates and many highly needed products can not be obtained in sufficient quantities.

Furthermore, a disadvantage of P450 enzymes in using them for industrial purposes is the requirement for the costly cofactor NADPH/NADH in the P450-catalyzed reactions. Not only are the cofactors very expensive, but they are also responsible for the inactivation of the enzymes when the concentration of substrates is low, resulting in incomplete oxidation of the substrates.

One possible enzyme of interest is the Old Yellow Enzyme (“OYE”). OYE are first known as enone reductase (ERED) on typical substrates such as cyclohexone and carvone.

The reactions commonly catalysed by OYE family enzymes range from asymmetric reaction of alpha, beta desaturated ketones (Hall, (2007) Angewandte) to the degradation of explosives (Williams, (2002) Microbiology).

In addition to the expected “enone” reduction, other enone reductases including the known OYE enzymes have been reported to catalyze the highly endothermic desaturation of C—C bonds, but only if such reactions are coupled with subsequent product aromatization (Vaz et al., 1995), as the energetically favorable product aromatization drives the desaturation reaction. However, the aromatized products do not retain the initial properties of the desaturated compound for follow up chemistry. No enzymatic introduction of double bonds which is not linked with the energetically favored product aromatization has been demonstrated thus far.

Thus there is a need for OYE enzymes that catalyze the hydroxylation of testosterone.

There is also a need for OYE enzymes that catalyze the desaturation reaction of ketones without subsequent product aromatization.

In general, heat stable enzymes have favorable properties for the use in biochemical reactions. Often heat stability is combined with good solvent stability and high total turnover numbers. Moreover, the heat stability can be used to facilitate the purification of the enzyme (e.g. with heat precipitation).

Accordingly, a need exists for improved enzymes, which could catalyze or mediate the reduction and/or oxidation of substrates of interest. Example of such substrates include testosterone, which can undergo hydroxylation and/or desaturation to yield important metabolites. In addition, there is a general need for heat stable enzymes for the afore-mentioned reactions.

The present invention addresses this need for an improved method and enzyme for the reduction and/or oxidation of substrates, without the disadvantages of conventional biocatalytic enzymes such as monooxygenases.

### SUMMARY OF THE INVENTION

The current invention provides a method of making a reduced substrate and/or an oxidized substrate using an isolated Old Yellow Enzyme.

The invention provides an isolated Old Yellow Enzyme capable of mediating the oxidation or reduction of a substrate into an oxidized and/or reduced substrate.

In one embodiment, the invention is directed to a method of the chemoselective and regioselective oxidation of carbon-hydrogen bonds using an isolated Old Yellow Enzyme

In one embodiment, the invention provides a method of hydroxylating testosterone using an isolated Old Yellow Enzyme.

In another embodiment, the invention provides an isolated Old Yellow Enzyme capable of hydroxylating testosterone.

In another embodiment, the invention provides a method of controlling the stereospecificity of the enzyme-mediated oxidation by utilizing hydrogen peroxide.

In another embodiment, the invention provides a method of oxidizing ketones to alpha, beta desaturated ketones using an Old Yellow Enzyme (e.g., introduction of an additional double bond in testosterone).

In another embodiment, the invention provides an isolated Old Yellow Enzyme capable of oxidizing ketones to alpha, betaalpha, beta desaturated ketones (e.g., introduction of additional double bonds in testosterone).

In another embodiment, the OYE-catalyzed desaturation of ketones proceeds without the need of energetically-favored subsequent product aromatization.

In another embodiment, the OYE-catalyzed desaturation of ketones proceeds without the addition of any coenzymes such as, e.g., NAD+, NADP+, NADH and NADPH.

In another embodiment, the invention is directed to an isolated OYE capable of hydroxylating testosterone in the presence of cofactor NADPH and catalyzing the desaturation of testosterone in the absence of any coenzymes such as, e.g., NAD+, NADP+, NADH and NADPH.

In t another embodiment, the invention is directed to an isolated OYE possessing high heat stability. Its heat stability facilitates enzyme purification, enhances storage stability, and allows higher reaction temperatures, long-term conversions, and/or enzyme recycling.

In another embodiment, the invention is directed to an isolated OYE capable of mediating the reduction of substrate stereoselectively at reaction rates higher than other known reductases.

### BRIEF DESCRIPTION OF THE DRAWINGS

These and other features, aspects, and advantages of the present invention will be more fully understood when considered with respect to the following detailed description, appended claims, and accompanying drawings, wherein:

FIG. 1 is a reaction diagram of the hydroxylation of testosterone to 6α and 6β-Hydroxytestosterone;

FIG. 2 is a Table showing the alignment of Geobacillus kaustophilus OYE with the homolog protein from Bacillus subtilis (GI: 67464349). The “query” is the sequence of the Geobacillus kaustophilus OYE, and the subject is the sequence of the YqjM;

FIG. 3 shows the OYE expressed in DH5a cells as a 38 kDalton band, which is absent in the negative control lane;

FIG. 4 is a reaction diagram of the desaturation of testosterone to boldenone;

FIG. 5 shows the conversion of cyclohexanone into Cyclohex-2-enone and further to phenol;

FIG. 6 shows the conversion of dihydrocarvone to carvone;

FIG. 7 shows the influence of exogenous hydrogen peroxide on the stereospecificity of the OYE mediated hydroxylation reaction;

FIG. 8 shows the OYE-mediated testosterone conversion: a) desaturation to boldenone; b) hydroxylation to 6-hydroxytestosterone;

FIG. 9 shows the isolation of the GkOYE by heat precipitation;

FIG. 10 shows the Cyclohexenone reduction by G. kaustophilus OYE and YqjM at different temperatures;

FIG. 11 shows the ¹H-NMR spectrum of 6α-hydroxytestosterone;

FIG. 12 shows the ¹H-NMR spectrum of 6β-hydroxytestosterone;

FIG. 13 shows the OYE expressed in different E. Coli expression strains as 38 kDalton bands; and

FIG. 14 shows the testosterone conversion into 6α-hydroxytestosterone as measured by HPLC-MS

### DETAILED DESCRIPTION OF THE INVENTION

The isolated OYE, according to the invention, is preferably extracted from the organism, and used either in raw lysates or in purified form.

In an enzymatic or enzyme-catalyzed reaction, according to the invention, the enzyme mediates the reaction by chemically utilizing the residues in its active. In contrast, in an enzyme-mediated reaction, the enzyme mediates the reaction without utilizing its active site, e.g., the substrate is activated by binding to the enzyme but no residues of the enzyme are involved in the reaction itself. According to the invention, the term “mediate a reaction” includes both enzyme-catalyzed and enzyme.

Thus, in a first aspect, the invention provides a method of making a reduced substrate and/or an oxidized substrate using an enzymatic or enzyme-mediated reaction, comprising contacting an isolated Old Yellow Enzyme (OYE) with a substrate to form a reaction product comprising a reduced substrate and/or oxidized substrate. In one embodiment, the invention provides a method for enzyme-mediated hydroxylation of testosterone into 6α and/or 6β hydroxytestosterone. In another embodiment, the invention provides a method for enzyme-mediated desaturation of testosterone to form desaturated testosterone.

In another aspect, the invention provides a method of oxidizing testosterone, comprising contacting testosterone with an isolated Old Yellow Enzyme (OYE) to form 6α and/or 6β-hydroxytestosterone. In another embodiment, the invention provides a method for further comprising controlling the ratio of 6α to 6β-hydroxytestosterone by contacting the isolated OYE with the substrate in the presence of hydrogen peroxide.

In another aspect, the invention provides a method of making an oxidized product from a ketone, comprising contacting the ketone with an isolated Old Yellow Enzyme (OYE) to form an alpha, beta desaturated ketone. In another embodiment, the alpha, beta desaturated ketone is formed without subsequent energetically favored product aromatization. In another embodiment, the alpha, beta desaturated ketone is formed in the absence of the nicotinamide cofactors and in the presence of molecular oxygen. In another embodiment, the ketone is testosterone.

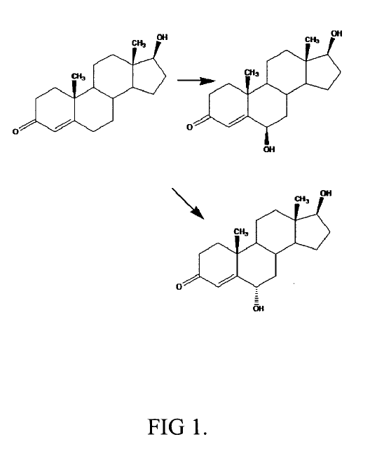

In the course of screening for new microbial hydroxylating activities, two strains are identified, both oxidizing testosterone to 6α and 6β hydroxytestosterone (FIG. 1). The two strains correspond to Geobacillus thermoglucosidasius and Geobacillus kaustophilus. There are also literature reports about P450 enzyme(s) from Geobacillus that can catalyze or mediate the hydroxylation of testosterone.

Protein purification from Geobacillus thermoglucosidasius and Geobacillus kaustophilus crude lysates and subsequent fingerprinting by Matrix-Assisted Laser Desorption/Ionization-Time-Of-Flight (MALDI-TOF) are chosen as a strategy for identifying the testosterone hydroxylating activity. To identify the enzyme responsible for the hydroxylating activity, Geobacillus thermoglucosidasius DSM 2542 and Geobacillus kaustophilus DSM 7263, the latter one having the advantage of a sequenced genome, are obtained from Deutsche Sammlung von Mikroorganismen und Zellkulturen GmbH (DSMZ; German Collection of Microorganisms and Cell Cultures) and tested for their ability to hydroxylate testosterone.

To purify the enzyme of interest, the raw lysates of the strain Geobacillus kaustophilus DSM 7263 are subjected to anion exchange chromatography followed by size exclusion chromatography. Several active fractions are obtained and loaded onto a Sodium Dodecyl Sulfate Polyacrylamide Gel Electrophoresis (“SDS-PAGE”) and, after gel electrophoresis, stained with Coomassie blue. One lane yields a single band of approximately 38 kDa, which is also present in all other active fractions. This band is isolated from the gel and prepared for fingerprinting by MALDI-TOF. A database search with the acquired fingerprint yielded, amongst others, OYE of Geobacillus kaustophilus, YP_{—}148185, referred to hereinafter as GkOYE.

Comparing the sequence of the active purified protein YP_{—}148185.1 against the genome sequence of Geobacillus kaustophilus facilitates the identification of the gene coding for YP_{—}148185.1.

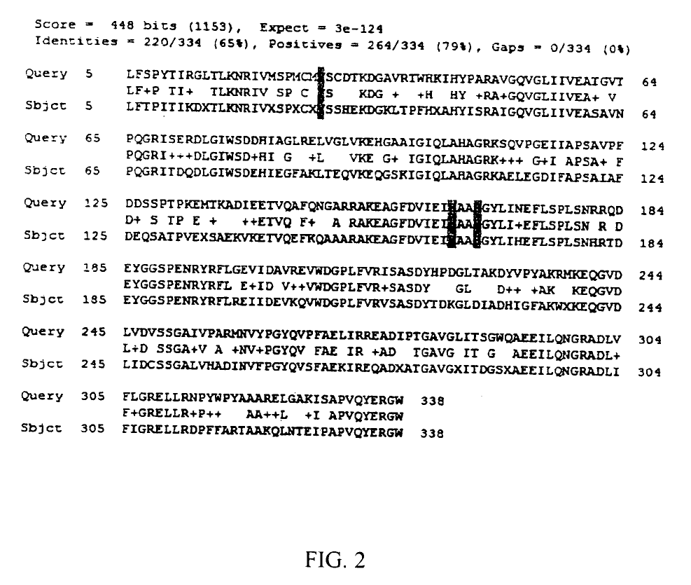

To identify homology with other known OYEs, a Basic Local Alignment Search Tool (BLAST) is used, which compares the DNA/protein sequences of the GkOYE with sequences of other known OYEs. The results of the search reveal close homology to the YqjM type of OYEs. The highest similarity is found with the YqjM sequence from Bacillus subtilis NRRL B-14911. The alignment establishing the similarity of the GkOYE to the homolog protein from Bacillus subtilis (GI: 67464349) whose the structure is known is shown in FIG. 2.

As shown in FIG. 2, the two Histidines in the substrate binding site, H164 and H167, are conserved. The Y28 of the N-terminal part, which is a special feature of the YqjM-like proteins and acts as a hydrogen donor, is also conserved. It should be noted that, in most OYEs, this residue Y28 is formed by a tyrosine from the C-terminal domain.

The cofactor involved in the GkOYE-catalyzed oxidation is determined to be FMN, and not FAD, by MALDI-TOF. Absorbance spectra of the oxidized enzyme show characteristic bands at 360 and 455 nm, with the latter peak showing an OYE characteristic fine spectrum (shoulders at 430 and 485 nm respectively). Also, the shorter wavelength peak shows a fine spectrum with a second maximum at 380 to 385 nm, which is not common for OYEs. Denaturation of the enzyme with 0.5% SDS shows a characteristic spectrum of free FAD/FMN, indicating that the cofactor is not bound covalently.

Primers are designed to amplify OYE from Geobacillus kaustophilus genomic DNA by polymerase chain reaction (PCR). PCR products yield a band at the expected size of approximately 1020 bp. The fragment is cloned into an expression vector (pEamTA), yielding pEamTAOYE. The sequence of the expressed protein confirmed that the cloned fragment is indeed the desired OYE.

For further expression of OYE, the DNA fragment of interest is transformed into a DH5α strain of E. coli using transformation procedures that are well known in the art.

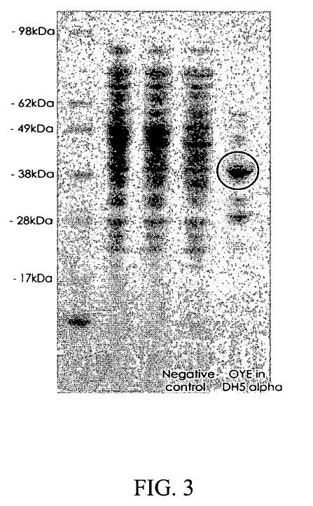

After expression in E. coli DH5α, the cells are harvested, ruptured, and centrifuged, and loaded onto a SDS-PAGE. A thick band, visible in the soluble fraction at the expected size of 38 kDa is observed. A small amount of OYE is also found to remain in the insoluble fraction. A negative control (pEamTA in DH5α) does not show a band at 38 kDa (FIG. 3).

The hydroxylation activity is preferably determined by measuring the oxidation of testosterone (5 mM), in the presence of NADPH (0.5 mM), into 6α-hydroxytestosterone at 55° C. High-Performance Liquid Chromatography/Mass Spectrometry (“HPLC/MS”) analysis is used to detect the production of 6α-hydroxytestosterone after 24 h, and a further increase in product yield after 48 h. After 48 h the reaction was stopped.

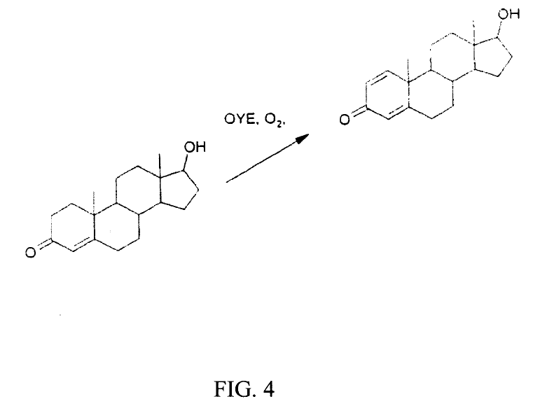

In addition to the hydroxylation activity, surprisingly, the Geobacillus kaustophilus OYE also catalyzed an O₂-driven, nicotinamide-independent desaturation reaction, introducing C—C double bonds adjacent to carbonyl groups. As shown in FIG. 4, GkOYE catalyzes the desaturation of testosterone in boldenone.

The GkOYE-catalyzed desaturation activity is preferably determined by measuring the oxidation of testosterone (5 mM) in the absence of cofactor NADPH into boldenone at 55° C. and incubated for incubated for 48 h. HPLC/MS analysis is used to confirm the production of desaturated testosterone. At a temperature of 70° C. and at a testosterone concentration of 1 mM, the yield of the reaction is above 90% after 48 h.

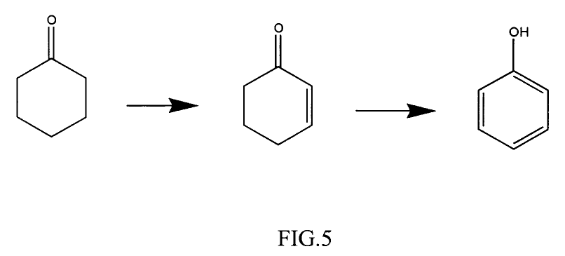

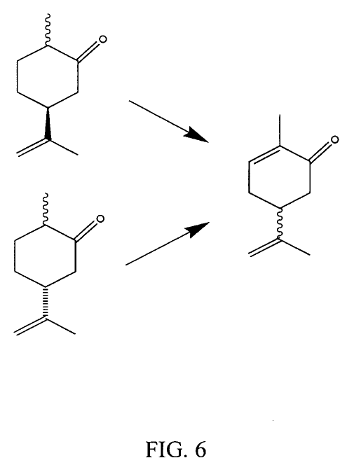

Furthermore, GkOYE also acts as “enone” reductase and mediates the reduction of typical substrates such as cyclohexenone and carvone. Interestingly, it is also capable of mediating the reverse reaction, the desaturation of cyclohexanone into Cyclohex-2-enone and further into phenol, as shown in FIG. 5. It should be noted that the dismutation reaction has been described in “Old Yellow Enzyme: Aromatization of Cyclic Enones and the Mechanism of a Novel Dismutation Reaction,” Alfin D. N. Vaz,* Sumita Chakraborty, and Vincent Massey. However, to date, cyclohexanone has not been reported as a starting material in such a desaturation reaction. The ratios of cyclohexanone:cyclohex-2-phenone:phenol after a 24 hour reaction are 60%:7%:30%.

Similarly, GkOYE is capable of catalyzing the desaturation of dyhydrocarvone into carvone, as shown in FIG. 6. At 70° C., both the R and S enantiomers of dihydrocarvone can be desaturated to carvone. The described desaturation reactions, however, are not temperature dependent, as the desaturation at 37° C. is comparable to the desaturation performed at higher temperatures (45, 50, 60, and 70° C.). At higher temperatures, the desaturation reaction is not stereoselective. The reverse reaction, reduction of carvone to dihydrocarvone, yields ˜50% of each R and S-(+) enantiomers.

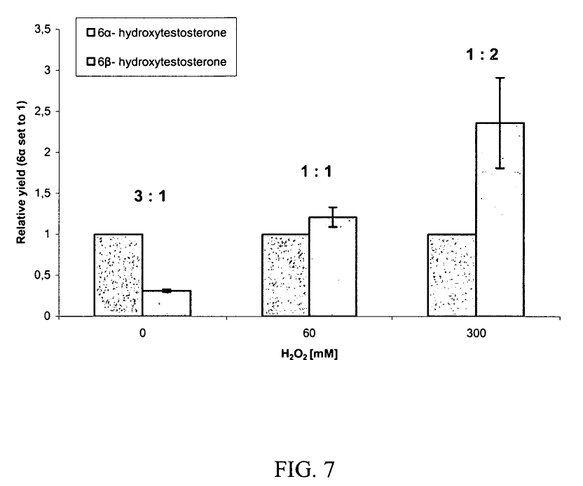

Addition of exogenous hydrogen peroxide induces a change in the stereoselectivity of GkOYE. In hydroxylation assays as described above and carried out in the presence of various concentrations of hydrogen peroxide (0.05-0.6%) (FIG. 7), it is found that hydrogen peroxide changes the stereoselectivity of the GkOYE-mediated hydroxylation of testosterone. As shown in FIG. 7, in the presence of reduced nicotinamide cofactor, testosterone is regioselectively hydroxylated at position 6, yielding 6α- and 6β-hydroxytestosterone, with a product ratio of approximately 3:1. Increasing hydrogen peroxide concentrations, however, inverts the ratios of 6α- and 6β-hydroxytestosterone, yielding a 1:2 ratio. Higher concentrations of H₂O₂ result in at least 4 additional hydroxylation products that remain to be identified.

Since the ratio of 6α-hydroxytestosterone to the human main metabolite, 6β-hydroxytestosterone, is influenced by varying hydrogen peroxide concentrations, testosterone hydroxylation appears to be enzyme-mediated rather than enzyme-catalyzed and involves the reduction of molecular oxygen to a reactive oxygen species at the flavin's isoalloxazine ring system. In an enzyme-mediated reaction, the enzyme facilitates the reaction without direct participation of the residues in the active site. In an enzyme-catalyzed reaction, the residues of the active site are physically changed.

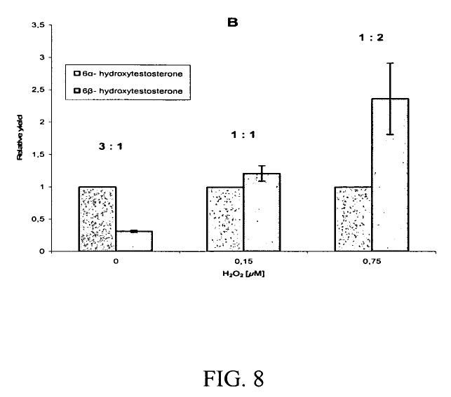

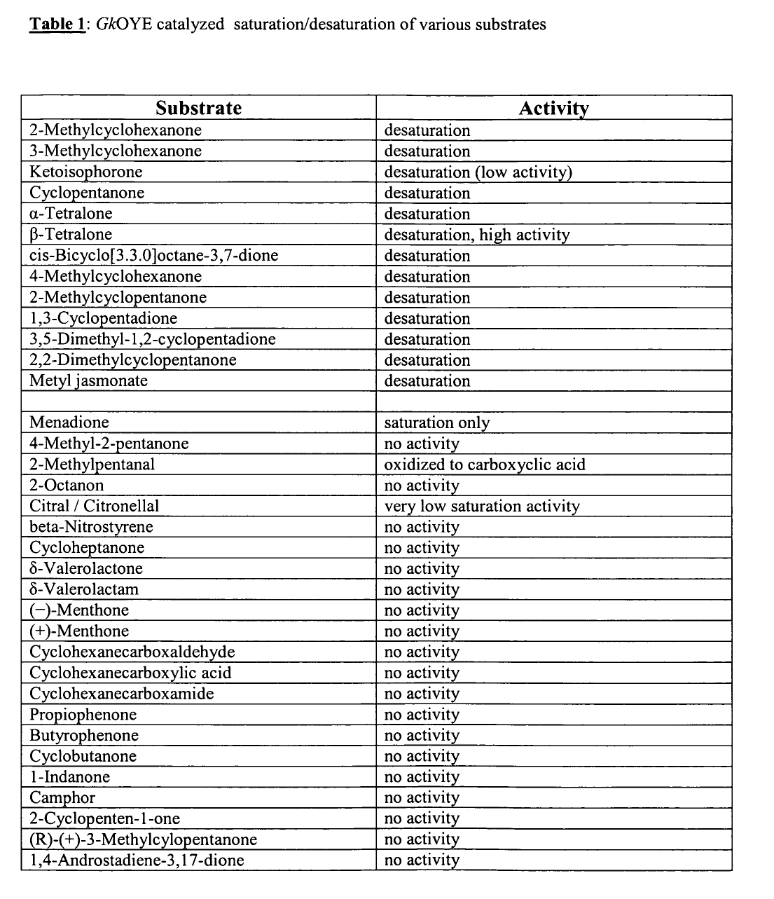

The hypothesis that the testosterone hydroxylation is mediated by GkOYE is substantiated by the observation that the mutation of the two Histidine residues at the active site to Alanine (H164A and H167A) destroys the saturation/desaturation activities but only slightly reduces hydroxylation activity. These results suggest that GkOYE mediates the hydroxylation of testosterone and also catalyzes desaturation of testosterone. FIG. 8 shows the GkOYE selective oxidation of testosterone depending on the presence or absence of cofactor NADPH and oxygen.

Yields of hydroxylation might be improved by reaction engineering. A sufficient supply of reduced cofactor (e.g. NADPH) should prevent desaturation of testosterone to boldenone and thus allow the hydroxylation of testosterone to 6α- and 6β-hydroxytestosterone.

Although not to be bound by theory, the results point to an enzyme-mediated process for the hydroxylation of testosterone. “Good” substrates are readily reduced, while “bad” substrates are bound, oriented, and slightly activated. The reduced Flavin cofactor (FMN) may transfer the electrons to molecular oxygen, and the resulting hydrogen peroxide could then hydroxylate the bound substrate. It is also possible that the oxidation reaction only takes place under higher temperatures, but this is hard to prove since all other available OYEs quickly precipitated at temperatures above 40° C.

Several substrates other than testosterone were also tested for desaturation activities (Table 1). The results show that GkOYE catalyzes the desaturation/saturation of several substrates, and confirm that OYEs can be used to facilitate the biooxidation of substrates. It has been further discovered that the use of an OYE allows for the production of oxidized substrates in one-step reactions at high yield. In addition, the GkOYE shows stability at high temperature and in long lasting bioconversions.

For example, in one experiment, where the enzyme's desaturation activities are measured as a function of temperature, the temperature was varied by 2.5 degree increments, from 30 to 85° C., and the optimum temperature for testosterone desaturation was found to be 70° C.

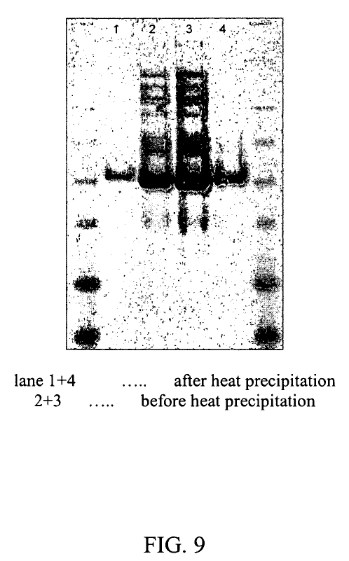

The thermo stability of GkOYE facilitates its purification by heat precipitation, as illustrated by example (give number of example) and as shown in FIG. 9. Activity measurements showed that no loss of activity after incubation at 55° C. for 10 min. Moreover, under these conditions most E. coli proteins precipitate, leaving the OYE in the supernatant in high purity. Even after one week at 4° C. no decrease in activity was observed.

The thermo stability in the bioxidation reactions seems to be unique to GkOYE and is not exhibited by other recombinant OYEs obtained by using commercial kits from Codexis, (Pasadena, Calif.) For example,

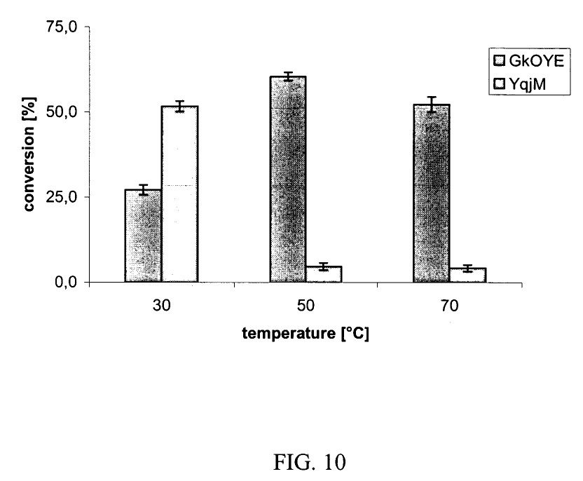

No testosterone hydroxylation or desaturation is observed with a range of recombinant OYEs from various other species (commercial kit from Codexis, Pasadena) when activity was measured as a function of temperature, over a temperature range of 30-70° C., varied in 5° C. increments, Even with the highly homologous Bacillus subtilis YqjM (similarity: 80%), no oxidized testosterone derivative is detected, even though enone reductase activity is demonstrated at 70° C. (FIG. 10). Furthermore, compared to other described enzymes, GkOYE exhibits a higher reaction rate for many common saturation substrates (e.g. Cyclohexenone).

The following are non-limiting examples of the invention.

### EXAMPLE 1

### Hydroxylation of Testosterone by Cell Lysates of Geobacillus thermoglucosidasius DSM 2542 and Geobacillus kaustophilus DSM 7263

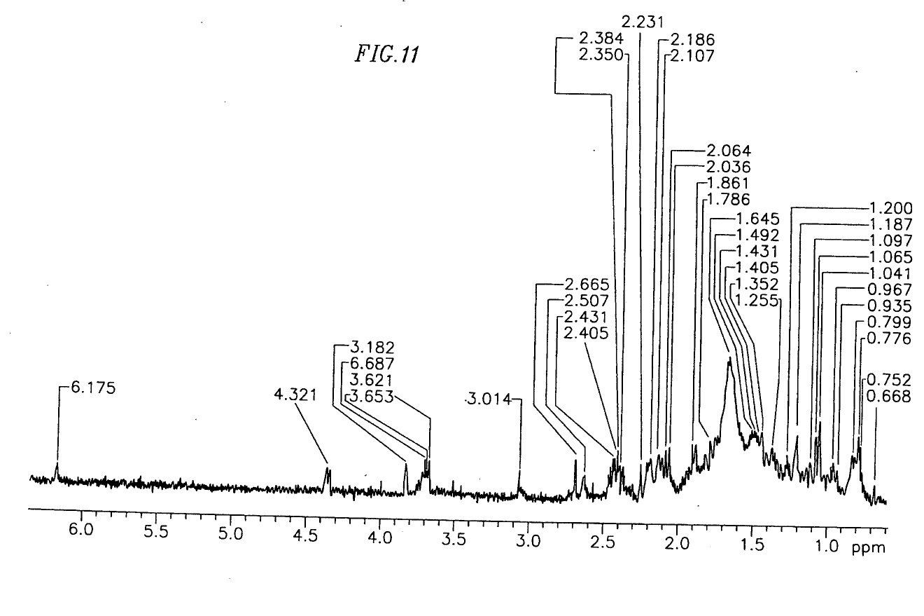

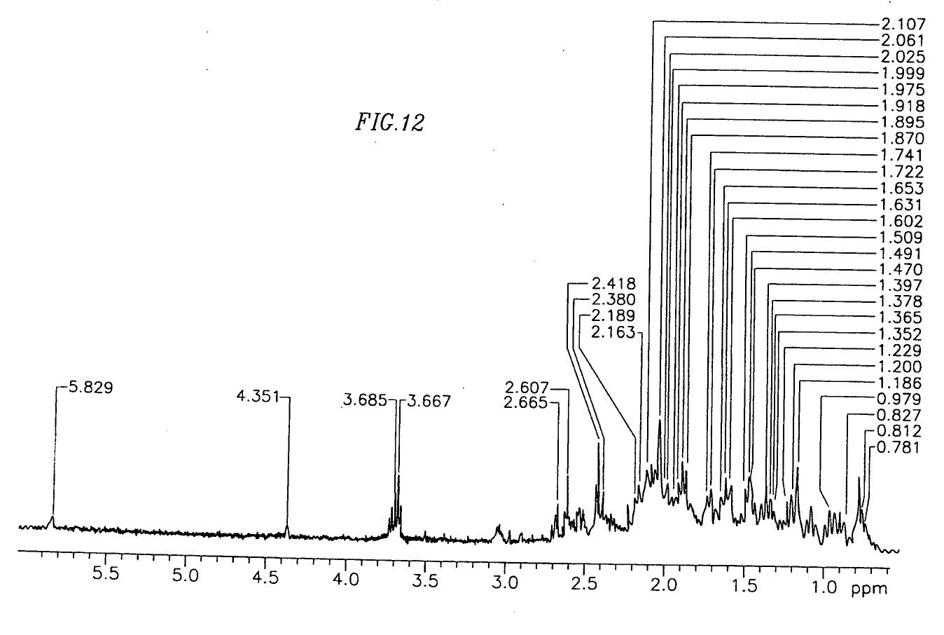

Geobacillus thermoglucosidasius DSM 2542 and Geobacillus kaustophilus DSM 7263 were obtained from Deutsche Sammlung von Mikroorganismen und Zellkulturen GmbH (DSMZ; German Collection of Microorganisms and Cell Cultures) and tested for their ability to hydroxylate testosterone. Raw lysates of both strains were analyzed by HPLC/MS and showed conversion to two products, both of which showed a m/z ratio of 305, as expected for hydroxylated testosterone, but with different retention times. While one of the metabolites corresponded exactly with an authentic 6β-hydroxytestosterone standard, the hydroxylation position of the second metabolite was unclear in the beginning. Both metabolites were then prepared on a milligram scale for ¹H-NMR analysis. The NMR spectra are shown in FIGS. 11 and 12. Peak 1 in FIG. 11 was identified as 6α-hydroxytestosterone and Peak 2 of FIG. 12 was identified as 6β-hydroxytestosterone.

### EXAMPLE 2

### Expression of OYE in Different Strains of E. Coli

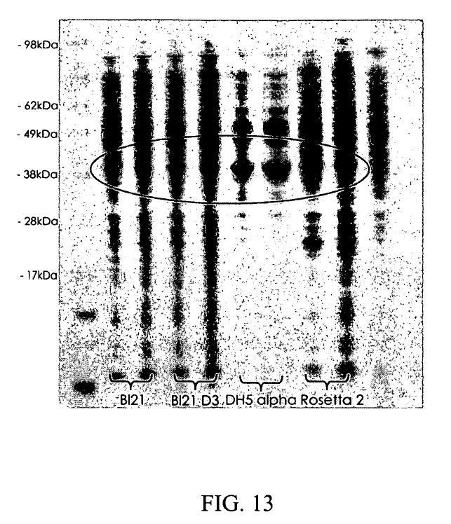

For further expression of OYE, new expression strains of E. Coli were chosen. 2 μL of OYE-DNA were transformed in the strains listed below.
Rosetta
Rosetta 2
Bl21
Bl21 D3
DH5α
   All cells except for DH5α were electrocompetent cells according to transformation procedures that are well known in the art. The regenerated cell suspension were plated out on LB-Amp-plates (100 μg/mL), except for the cells of the Rosetta strains, which were plated on LB-AMP-Chloramphenicol plates (100 μg/mL). After an incubation period of about 24 hours at 37° C., the grown colonies were used for inoculation of 100 mL LB-Amp and LB-AMP-Chloramphenicol, respectively. No growth on agar plates after transformation was recorded for the Rosetta cells, and almost no colonies had been obtained by using the Rosetta 2 cells. The inoculated flasks were shaken constantly with 120 rpm at 28° C. After 5 hours at an optical density of 0.5, protein expression was induced by adding 0.5 mM IPTG to each flask. The temperature was then lowered to 20° C. The cells were harvested, ruptured, and ultracentrifuged, and the supernatants were loaded on SDS-PAGE. FIG. 13 shows the expression of OYE in different expression E. coli strains. Using DH5α cells for expression of OYE gave the best results, followed by Rosetta 2.

### EXAMPLE 3

### Expression of OYE by DH5α Cells

As a result of the limited number of transformants by using the Rosetta 2 cells, for further expression procedures of OYE only the chemical competent DH5α cells were used. FIG. 3 shows the expression of OYE in DH5α cells only. DH5α cells transformed with the vector pEamTA (without the OYE fragment) were used as a negative control. A 38 kDalton band was obtained in the OYE transformed DH5α, but was absent in the negative control.

### EXAMPLE 4

### Hydroxylation Activity of the OYE-Containing Fraction

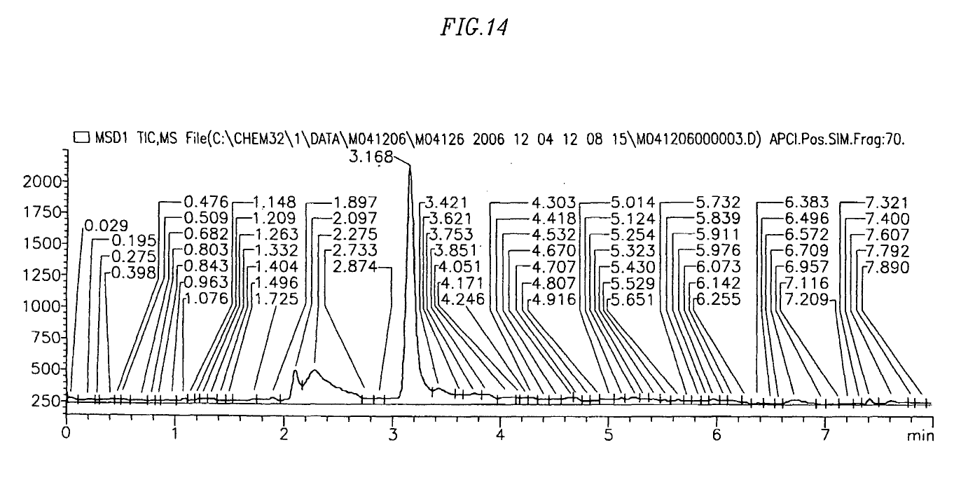

To verify that the fraction that yielded a 38 kDalton band on the gel as shown in FIG. 3 contains hydroxylating capability, the fraction was tested for hydroxylation activity wherein the testosterone conversion was analyzed by HPLC-MS. FIG. 14 shows a peak corresponding to the formation of 6α-hydroxytestosterone.

### EXAMPLE 5

### Oxidation of Testosterone as Catalyzed or Mediated by GkOYE in the Presence or Absence of Cofactors at Various Temperatures

The following stock solutions were prepared:

| | |
|---|---|
| protein solution | 20 mg/mL, 50 mM KPi, pH = 7 |
| NADPH | 10 mM, 50 mM KPi, pH = 7 |
| Testosterone | 100 mM, EtOH |
| GDH | 10 mg/mL GDH102, 50 mM KPi, pH = 7 |
| Glucose | 200 mM, , 50 mM KPi, pH = 7 |

All samples were filled up to a total volume of 1 mL with 50 mM KPi, pH=7

| Sample | Composition | Volume | |
|---|---|---|---|
| 1 | protein solution | 500 | μL |
| | NADPH | 100 | μL |
| 2 | protein solution | 500 | μL |
| | testosterone | 10 | μL |
| 3 | protein solution | 500 | μL |
| | testosterone | 10 | μL |
| | NADPH | 100 | μL |
| 4 | protein solution | 500 | μL |
| | testosterone | 10 | μL |
| | NADPH | 100 | μL |
| | GDH | 100 | μL |
| | Glucose | 300 | μL |
| 5 | testosterone | 10 | μL |
| | NADPH | 100 | μL |
| | GDH | 100 | μL |
| | Glucose | 300 | μL |

The reactions were carried out at 37, 50 and 70° C. It was found that no reaction occurred with the negative controls (samples 1+5). No reaction was observed at the lowest temperature 37° C. Starting at 50° C. without any cofactor, the main metabolite had a reaction time of 9.9 min and a molecular weight (MW) of 286 determined by HPLC-MS. Compared to testosterone this is a reduction of 2 mass units, as expected for the desaturation reaction. Samples 3 (with NADPH) and 4 (with GDH cofactor regeneration) showed reduced formation of the desaturation product, most likely because NADPH competes with the testosterone to reduce the enzyme. Follow-up reactions indicate that oxygen is reoxidizing the enzyme (see Example 6: anaerobic testosterone desaturation). After 48 h, sample 2 showed 84% conversion. After 120 h, all testosterone was consumed. Sample 4 showed two peaks at ˜5 min corresponding to 6α—(7%) and 6β—(6%) hydroxytestosterone. LC-MS showed an exact mass of 304. After further incubation, desaturation of the hydroxylated products occurred, yielding products with MW of 302.

### EXAMPLE 6

### Anaerobic Testosterone Desaturation

To support the hypothesis that molecular oxygen is responsible for reoxidizing the enzyme, protein solution and substrate (for composition see sample 2 in Example 5) were incubated for 48 h in a minimal oxygen environment. Oxygen was removed by applying vacuum for 15 min followed by flushing with Nitrogen (three rounds) in a sealed glass tube. The reaction was incubated at 70° C. for 48 h. Under aerobic conditions, this reaction yielded 84% desaturation product, but under anaerobic conditions only 18% desaturated testosterone was formed, supporting the assumption that molecular oxygen acts as the final electron acceptor in this reaction.

### EXAMPLE 7

### Desaturation of Cyclohexanone by GkOYE

The reaction sample contained 500 μL protein solution and 10 mM Cyclohexanone (˜1 μL). The final volume of the reaction was ˜500 μL. The reaction was carried out at 70° C. for 24 hours. It was found that Cyclohexanone was desaturated to Cyclohex-2-enone and further to phenol. After 24 hours, the yields of the products were as indicated in FIG. 5.

### EXAMPLE 8

### Oxidation/Reduction of Dihydrocarvone/Carvone by GkOYE

The sample for the oxidation reaction contained 500 μL protein solution and 10 mM (R,S)-(+) Dihydrocarvone (˜1.5 μL). The final volume of the reaction was 500 μL. The reaction was carried out for 24 h at varying temperatures (see table 2).

The sample for the reduction reaction contained: 500 μL protein solution+(+)Carvone (˜1.5 μL)+200 μL GDH 102 (10 mg/mL) and Glucose (200 mM)+100 μL NADPH (10 mM). The final volume of the reaction was 800 μL. The reaction was carried out for 24 h at various temperatures (see table 2).

It was found that, regardless of which starting enantiomer was used, dihydrocarvone was reduced to carvone. Moreover, the reverse reaction yielded ˜50% of each, R and S-(+) Dihydrocarvone (also done at 70° C.). The desaturation reaction was also carried out at different temperatures: 37, 45, 50, 60 and 70° C. Desaturation was observed even at 37° C. in comparable yields to those seen at higher temperatures. At higher temperatures, however, the reaction was not stereoselective.

### EXAMPLE 9

### Hydroxylation and Desaturation Activities of GkOYE Mutants

Mutants of GkOYE (Y28F, H164A, H167A; H164A/H167A; 2 clones each) were transformed into and expressed in E. coli DH5alpha. SDS-Page revealed good expression levels for all clones. Conversions of testosterone at 55° C. with NADPH and 70° C. without the cofactor showed slightly reduced hydroxylation activity for all mutants and, surprisingly, desaturation activity for Y28F (Y28 is assumed to be the proton donor for Yqjm saturation reaction) but not for H164A and H167A or the double His mutant. Titration of the Mutants with pHBA revealed a K_{d} in the mM range (1-10 mM), which is approximately a factor 10³ worse than 3.1 μM of the wild type enzyme.

### EXAMPLE 10

### Heat Precipitation of GkOYE

The reaction sample contained the following:

buffer: 50 mM Kpi; pH=7
10 min, 55° C.;
heating: boiling hot water
cooling: ice water
The Activity was tested with NADPH depletion assay before and after heat precipitation and after lyophilization of purified enzyme:

| | |
|---|---|
| before heat precipitation: | 1.14 U/mg Lyo |
| after heat precipitation: | 1.90 U/mg Lyo |
| after lyophilization: | 2.16 U/mg Lyo |

The Protein concentration was determined as 84% of Lyo by Bradford assay.
Lanes 1 and 4 of FIG. 9 show the enzyme after heat precipitation and lanes and 2 and 3 before the heat precipitation.

### EXAMPLE 11

### Heat Precipitation of GkOYE

E. coli DH5alpha harbouring pEamTAGkOYE was cultivated in a 5 L bioreactor. The enzyme was purified by heat precipitation and yielded ˜45 g of lyophilisate (>8 g/L purified enzyme). The stereoselectivity of GkOYE saturation was tested by reducing R-(−)-carvone to dihydrocarvone (50° C.): yield>99%; ee (enantiomeric excess)=75%, NOE-NMR experiments with the derivatized product showed that the preferred product is (2R,5R)-Dihydrocarvone.

### EXAMPLE 12

### Thermostability of GkOYE Relative to Other Recombinant OYEs

Expression clones of YqjM (B. subtilis) and OPR3 (tomato) were obtained from Peter Macheroux, Institute of Biochemistry, TU-Graz. The reductase activities of Yqjm and OPR3 were confirmed using cyclohexenone and carvone as substrates. Both showed stereoselectivity comparable to GkOYE. The desaturation reaction was carried out using cyclohexanone with all three enzymes at 30, 50, and 70° C. The reductase activity using cyclohexenone as substrate was also measured under similar conditions. The results showed that, while OP3 was almost inactive at 50° C., YqjM showed a significantly reduced reductase activity up to the highest temperature (70° C.). However, no desaturation was observed for both OP3 and YqjM enzymes. On the other hand, GkOYE as a positive control showed cyclohexane desaturation activity starting from 50° C. and saturation of cyclohexenone at all temperatures tested.

### EXAMPLE 13

### Expression of GkOYE by DH5α and Measurements of its Activities

Then following primers were used: GkOYEfw1: ATG AAC ACG ATG CTG; and GkOYErv: GAA TTC TTA TTA AAA CCG CCA GC. All oligonucleotides used were manufactured by Invitrogen. The following conditions were used for PCR amplification: 20 ng genomic DNA, digested with Not1, 1 μL primer GkOYEfw1 (10 pmol/μL), 1 μL primer GkOYErv (10 pmol/μL), 1 μL dNTP mix (10 mM), 10 μL 5× Colorless GoTaq Reaction Buffer, 1 μL GoTaq-Polymerase (Promega), filled up with ddH₂O to a total volume of 50 μL. Initial denaturation 3 min at 95° C., 25 cycles of 30 s at 95° C., 30 s at 42° C. and 90 s at 72° C., final elongation 10 min at 72° C. The amplified PCR product was purified using the WizardSV Gel and PCR clean-up system (Promega) and cloned into pEamTA (6) and sequenced, yielding pEamTAOYE.

E. coli DH5α harboring pEamTAOYE were used to express GkOYE. Cells were grown in an Infors incubator shaking at a diameter of 2.5 cm at 120 rpm and 37° C. At an OD₆₀₀ of 0.5, 1 mM IPTG was added and the incubation temperature was lowered to 30° C. After 24 h of expression, cells were harvested, lysed by sonication (50 mM KPi, pH 7), and E. coli proteins were precipitated at 55° C. for 10 min. Cell debris and precipitated host proteins were removed by centrifugation (8000×g, 15 min). The bright yellow supernatant was concentrated 10-fold using Vivaspin (Sartorius) ultrafiltration devices with a cutoff size of 10 kDa.

For spectral characterization, GkOYE was further purified via Anion exchange chromatography (QFF, GE healthcare) and size exclusion chromatography (Sephadex 75, GE healthcare).

For desaturation reactions, 500 μL protein solution (20 mg/mL) was added to a 10 mM substrate solution in 500 μL of a 50 mM KPi buffer, pH 7. Analysis of Dihydrocarvone/Carvone was done on a Shimadzu GC-17A with a Shimadzu GCMS-QP5050A Detector. The employed column was a XTI-5 from Restek (bonded 5% phenyl, length 30 m, thickness 25 μm, diameter 0.25 mm). High performance liquid chromatography/mass spectroscopy of Boldenone/Testosterone was done on an Agilent 1200 HPLC system with an UV detector and an Agilent G1956B mass detector. For separation, a Merck LiChroCART Purospher RP18 endcapped column with the dimensions 250 mm×4.6 mm×5 μm was employed. Mass spectra were recorded in positive ionization mode employing an APCI ion source.

Those skilled in the art will appreciate that the foregoing examples and descriptions of various preferred embodiments of the present invention are merely illustrative of the invention as a whole, and that variations in the methodology of the present invention may be made without departing from the invention. Accordingly, the present invention is not limited to the specific embodiments described herein but, rather, is defined by the appended claims and equivalents thereof.

TABLE 2
Reaction conditions and yields for Carvone/Dihydrocarvone.

| Reaction | Temp [° C.] | pH | Yield [%] | ratio A/B | Time [h] |
|---|---|---|---|---|---|
| oxidation | 70 | 7.0 | 61 | 75/25 | 24 |
| oxidation | 60 | 7.0 | 54 | 70/30 | 24 |
| oxidation | 50 | 7.0 | 21 | 77/23 | 24 |
| oxidation | 45 | 7.0 | 44 | 70/30 | 24 |
| oxidation | 37 | 7.0 | 26 | 75/25 | 24 |
| oxidation | 45 | 6.0 | 14 | 78/22 | 24 |
| oxidation | 45 | 8.5 | 42 | 76/24 | 24 |
| reduction | 70 | 7.0 | 88 | 48/52 | 24 |
| reduction | 37 | 6.0 | 97 | 48/52 | 24 |
| reduction | \|37\| | \|7.0\| | \|100\| | \|74/26\| | \|24\| |

What is claimed is:

1. A method of making a reduced substrate and/or an oxidized substrate using an enzymatic or enzyme-mediated reaction, comprising:
contacting an isolated Old Yellow Enzyme (OYE) with a substrate to form a reaction product comprising a reduced substrate and/or oxidized substrate.

2. The method of claim 1, wherein the reaction is a hydroxylation reaction.

3. The method of claim 1, wherein the substrate is testosterone and the reaction product is 6α and/or 6β-hydroxytestosterone.

4. The method of claim 1, wherein the reaction is a desaturation reaction.

5. The method of claim 1, wherein the substrate is a ketone and the reaction product is an alpha, beta unsaturated ketone.

6. The method of claim 1, wherein the substrate is testosterone and the reaction product is a desaturated testosterone.

7. The method of claim 1, wherein the substrate is dihydrocarvone and the reaction product is a desaturated dihydrocarvone.

8. The method of claim 1, wherein the substrate is cyclohexanone and the reaction product is a desaturated cyclohexanone.

9. The method of claim 1, wherein the isolated OYE catalyses the reverse reaction of enone reductases leading to alpha, beta unsaturated compounds.

10. The method of claim 9, wherein the reverse reaction is not coupled to product aromatization.

11. The method of claim 9, wherein the isolated OYE is contacted with the substrate in the presence of one or more cofactors selected from the group consisting of NAD+, NADH, NADP+, and NADPH.

12. The method of claim 9, wherein the isolated OYE is contacted with the substrate in the absence of nicotinamide cofactors.

13. The method of claim 1, wherein the oxidation or reduction of the substrate is a one-step reaction.

14. The method of claim 1, wherein the reaction occurs at temperatures higher than 45° C.

15. The method of claim 1, wherein the reaction occurs at temperatures higher than 65° C.

16. A method of oxidizing testosterone, comprising:
contacting testosterone with an isolated Old Yellow Enzyme (OYE) to form 6α and/or 6β-hydroxytestosterone.

17. The method of claim 16, further comprising controlling the ratio of 6α to 6β-hydroxytestosterone by contacting the isolated OYE with the substrate in the presence of hydrogen peroxide.

18. A method of making an oxidized product from a ketone, comprising:
contacting the ketone with an isolated Old Yellow Enzyme (OYE) to form an alpha, beta desaturated ketone.

19. The method of claim 18, wherein the alpha, beta desaturated ketone is formed without subsequent energetically favored product aromatization.

20. The method of claim 18, wherein the isolated OYE is contacted with the substrate in the presence of one or more cofactors selected from the group consisting of NAD+, NADH, NADP+, and NADPH.

21. The method of claim 18, wherein the isolated OYE is contacted with the substrate in the absence of nicotinamide cofactors.

22. The method of claim 18, wherein the ketone is testosterone.

23. An isolated Old Yellow Enzyme (OYE) capable of mediating the oxidation or reduction of a substrate into an oxidized and/or reduced substrate.

24. The isolated OYE of claim 23, wherein the isolated OYE is capable of oxidizing testosterone to 6α and/or 6β-hydroxytestosterone.

25. The isolated OYE of claim 23, wherein the substrate is a saturated compound and the isolated OYE is capable of oxidizing the substrated to alpha, beta unsaturated compounds.

26. The isolated OYE of claim 25, wherein the alpha, beta unsaturated compounds are formed without subsequent energetically favored product aromatization.

27. The isolated OYE of claim 23, wherein the substrate is a ketone and the isolated OYE is capable of oxidizing the substrate to an alpha, beta desaturated ketone.

28. The isolated OYE of claim 23, wherein the substrate is testosterone and the OYE is capable of oxidizing the substrate to desaturated testosterone.

29. The isolated OYE of claim 23, wherein the isolated OYE is capable of using molecular oxygen for substrate oxidations without subsequent energetically favored product aromatization.

30. The isolated OYE of claim 23, wherein the isolated OYE has been purified by heat precipitation.

31. The isolated OYE of claim 23, wherein the isolated OYE is capable of mediating the reduction of the substrate stereoselectively at reaction rates higher than other known reductases.

32. An isolated Old Yellow Enzyme (OYE) capable of mediating hydroxylation of a substrate and/or oxidation of the substrate to its desaturated products.

33. The isolated OYE of claim 32, wherein the substrate is testosterone.

34. The isolated OYE of claim 32, wherein the substrate is testosterone and the isolated OYE is capable of hydroxylating the substrate to 6α and/or 6β-hydroxytestosterone.

35. The isolated OYE of claim 32, wherein the substrate is testosterone and the isolated OYE is capable of oxidizing the substrate to desaturated testosterone.

36. The isolated OYE of claim 32, wherein the isolated OYE mediates the hydroxylation of the substrate in the presence of one or more cofactors selected from the group consisting of NAD+, NADH, NADP+, and NADPH.

37. The isolated OYE of claim 32, wherein the isolated OYE mediates the oxidation of the substrate to its desaturated products in the absence of nicotinamide cofactors and in the presence of molecular oxygen.
